(19) United States
(12) Patent Application Publication
(10) Pub. No.: US 2010/0003742 A1
(43) Pub. Date: Jan. 7, 2010

(54) METHOD FOR REDUCING FUCOSE CONTENTS OF RECOMBINANT PROTEINS

(75) Inventors: Jung-Seob KIM, Yongin-si (KR); Jae-Hoon MOON, Gunpo-si (KR); Mee Sook OH, Yongin-si (KR); Kong Ju LEE, Yongin-si (KR); Yeup YOON, Gwacheon-si (KR)

Correspondence Address: ROTHWELL, FIGG, ERNST & MANBECK, P.C., 1425 K STREET, N.W., SUITE 800, WASHINGTON, DC, 20005, US

(73) Assignee: MOGAM BIOTECHNOLOGY RESEARCH INSTITUTE, Yongin-si (KR)

(21) Appl. No.: 12/351,239

(22) Filed: Jan. 9, 2009

(30) Foreign Application Priority Data
Jul. 3, 2008 (KR) 10-2008-0064483

(51) Int. Cl. C07K 1/14

(52) U.S. Cl. 435/272

(57) ABSTRACT

The present invention relates to a method for reducing the fucose content of a recombinant protein, which comprises expressing in an animal cell the recombinant protein and FUCA1, an FUCA1 mutant, FUCA2, or a fragment of FUT8 localization domain; or with a fusion protein of a fragment of FUT8 localization domain and a fragment of FUCA1, a FUCA1 mutant or FUCA2. Therefore, the antibody expressed according to the method of the present invention exhibits a reduced fucose content in their Fc regions, which leads to the improvement in the therapeutic effect thereof.

### CROSS REFERENCE TO RELATED APPLICATIONS

This application claims priority from Korean patent application No. 10-2008-0064483 filed on Jul. 3, 2008, all of which is incorporated herein by reference in its entirety for all purposes.

### FIELD OF THE INVENTION

The present invention relates to a method for reducing the fucose content of a recombinant protein, which comprises expressing in an animal cell the recombinant protein and FUCA1, an FUCA1 mutant, FUCA2, or a fragment of FUT8 localization domain; or with a fusion protein of a fragment of FUT8 localization domain and a fragment of FUCA1, a FUCA1 mutant or FUCA2.

### BACKGROUND OF THE INVENTION

With the completion of human genome project and the identification of numerous disease-related genes through post-genomic approaches, therapeutic protein research has undergone an enormous progress. Most therapeutic proteins are glycoproteins whose therapeutic effects had been previously known to be affected only by their amino acid sequences. However, it has recently been established that the activity of a therapeutic protein can be effectively improved by modifying the sugar chain attached thereto, and such a sugar chain has been reported to be involved in variable biological functions including organogenesis, aging, infection, inflammation, biodefense, oncogenesis, cancer metastasis, tissue degeneration, regeneration and apoptosis. Glycomics is a glyco-engineering technology devoted to improve the therapeutic effect of conventional medicines by modifying its sugar chain so as to elevate both the activity and the stability, and also to minimize the effective dose and the adverse effects of them.

After the introduction in 1986 by Ortho Biotech Inc. of the first therapeutic antibody ‘OKT3’, a murine-derived antibody for the treatment of kidney transplant rejection, Centocor Inc. has developed ‘Reopro’ for inhibiting a platelet aggregation and ‘Remicade’ for treating Crohn's disease in 1994; and Genentech Inc., ‘Herceptin’ for treating breast cancer and ‘Rituxan’ for treating non-Nodgkins lymphoma (NHL). The development of the therapeutic antibody has been pursued in the fields of (i) technologies for the construction of chimeric, humanized and human antibodies (Jain, M. et al., Trend in Biotechnology, 25(7), 307-316, 2007), (ii) technologies for increased therapeutic effects of antibodies using Fc engineering (Lazar, G. A. et al., PNAS, 103(11), 4005-4010, 2006) or glycomics (Shuster, M. et al., Cancer Research, 65(17), 7934-7941, 2005; and Yamane-Ohnuki, N. et al., Biotechnology and Bioengineering, 87(5), 614-622, 2004), and (iii) technologies for enhanced efficiency in the production and purification of antibodies (Roque, A. C. et al., Biotechnology Progress, 20(3), 639-654, 2004).

The activity of a therapeutic antibody depends on its interaction with the Fc region receptor (FcrRIII) of natural killer (NK) cell, and it has been demonstrated that the removal of fucose from the sugar chain attached to Asn297 in Fc region of IgG leads to markedly strengthened interaction between the Fc region of IgG and Fc region receptor of NK cell, resulting in enhanced antibody-dependent cellular toxicity (ADCC) (Shields, R. L. et al., The Journal of Biological Chemistry, 277(30), 26733-26740, 2002).

Also, methods for elevating the ADCC of an antibody by reducing the fucose content in the sugar chain of the antibody have been disclosed, examples of which included methods for producing alpha 1,6-fucosyl transferase (FUT8) knock-out cell lines (Yamane-Ohnuki, N. et al., Biotechnology and Bioengineering, 87(5), 614-622, 2004; and U.S. Patent Publication No. 2004-0110704), methods for lowering the FUT8 expression using RNA interference (Mori, K. et al., Biotechnology and Bioengineering, 88(7), 901-908, 2004), and methods for inhibiting the expression of enzymes (e.g., GMD, Fx and GFPP) related to the synthesis of GDP-fucose which is a substrate for fucose synthesis (U.S. Patent Publication No. 2004-0093621).

Further, disclosed are method for reducing the fucose content by overexpressing N-acetylglycosaminyltrasnferase III (GnTIII) to inhibit the attachment of fucose to the sugar chain structure (Shuster, M. et al., Cancer Research, 65(17), 7934-7941, 2005, and U.S. Pat. No. 6,602,684) and a method for preparing a therapeutic antibody having enhanced ADCC by replacing the 239^{th} serine with aspartic acid, the 332^{nd} isoleucine with glutamic acid and the 330^{th} alanine with leucine, respectively, of the antibody Fc region so as to increase the interaction with Fc region receptor (Lazar, G. A. et al., PNAS, 103(11), 4005-4010, 2006).

Fucosidase represents a set of enzymes that remove fucose from the sugar chain of protein and others including carbohydrates through hydrolysis, and it can be devided into tissue fucosidase (alpha-L-fucosidase-1, FUCA1) and plasma fucosidase (alpha-L-fucosidase-2, FUCA2) according to its localization. FUCA1 is a lysosomal protein known to have low pH-triggering activity, while FUCA2 is a cell membrane or soluble protein which is activated under a neutral condition (Aviles, M. et al., Biochemical journal, 318, 821-831, 1996). However, there has been no attempt to modify antibody-expressing host cells by way of manipulating the activity of fucosidase, e.g., to make fucosidase localized in the Golgi apparatus involved in antibody secretion, so that fucose in the sugar chain structure can be effectively removed.

FUT8 is a type II integral membrane protein localized in the Golgi apparatus, which has C-terminal catalytic domain located in the lumen and N-terminal cytoplasmic tail (CT) (Milland, J. et al., The Journal of Biological Chemistry, 277(12), 10374-10378, 2002). The CT, the transmembrane domain (TM), and the stem region (stem) of FUT8 have been suggested to play a significantly concerted role in the protein localization and retention in the Golgi apparatus, but the exact mechanism of action has not known, e.g., whether these enzymes form a dimer or not in action (Nilsson, T. et al., The EMBO Journal, 13(3), 562-574, 1994). Further, there has never been disclosed to date a method for inhibiting the enzyme activity using the CT, TM or stem region.

Accordingly, the present inventors have endeavored to develop an effective and new method for reducing the fucose content in an antibody, and have found that the fucose content of an antibody can be markedly reduced and the therapeutic effect of the antibody can be improved by a method comprising making fucosidase or its derivatives localized in the Golgi apparatus or expressing a recombinant protein using a FUT8 localization domain (a domain including CT, TM and stem region).

### SUMMARY OF THE INVENTION

Accordingly, it is an object of the present invention to provide a method for reducing the fucose content of a recombinant protein, which comprises expressing in an animal cell the recombinant protein and FUCA1, an FUCA1 mutant, FUCA2, or a fragment of FUT8 localization domain; or with a fusion protein of a fragment of FUT8 localization domain and a fragment of FUCA1, a FUCA1 mutant or FUCA2.

In accordance with one aspect of the present invention, there is provided a method for reducing the fucose content of a recombinant protein, which comprises expressing in an animal cell the recombinant protein and one or more proteins selected from the group consisting of: a) FUCA1 having the amino acid sequence of SEQ ID NO: 6; b) an FUCA1 mutant having an amino acid sequence obtained by replacing asparagine of the amino acid sequence of FUCA1 with other amino acid; c) FUCA2 having the amino acid sequence of SEQ ID NO: 7; and d) a fragment of the localization domain of FUT8 having the amino acid sequence of SEQ ID NO: 1.

In accordance with another aspect of the present invention, there is provided a method for reducing the fucose content of a recombinant protein, which comprises expressing in an animal cell the recombinant protein and a fusion protein obtained by fusing a fragment of the localization domain of FUT8 having the amino acid sequence of SEQ ID NO: 1 with a protein selected from the group consisting of: a) a fragment of FUCA1, which has the amino acid sequence obtained by deleting 1^{st} to 26^{th} amino acids of the amino acid sequence of SEQ ID NO: 6; b) a fragment of FUCA2, which has the amino acid sequence obtained by deleting 1^{st} to 28^{th} amino acids of the amino acid sequence of SEQ ID NO: 7; and c) a mutant of FUCA1 fragment, which has the amino acid sequence obtained by replacing asparagine of the amino acid sequence of the fragment of FUCA1 with other amino acid.

### BRIEF DESCRIPTION OF THE DRAWINGS

The above and other objects and features of the present invention will become apparent from the following description of the invention, when taken in conjunction with the accompanying drawings, which respectively show:

FIG. 1: The gene structures of the modifying proteins obtained using FUT8 and/or fucosidase for the modification of the sugar chain;

FIG. 2A: Western blot analysis of CHO cells expressing glyco-modifying gene of FUT8, FUCA1 or FUCA2 mutant;

FIG. 2B: RT-PCR analysis for subdlones individually isolated from FUT8-stem1 CHO cells expressing glyco-modifying gene of FUT8-stem1;

FIGS. 3A and 3B: FACS analysis using LCA lectin showing fucose contents in CHO host cell lines containing sugar chain modifying genes;

FIG. 4: The fucose to four N-acetylglucosamine ratios determined by monosaccharides analysis using BIO-LC of antibodies produced in sugar chain-modified cell lines;

FIG. 5A: The analysis of complement-dependent cytotoxicitiy (CDC) of antibodies produced in sugar chain-modified cell lines;

FIG. 5B: The analysis of binding affinity of antibodies produced in sugar chain-modified cell lines with Fc gamma receptor IIIa (Fc RIIIa) expressed in CHO cell; and

FIG. 5C: The analysis of antibody-dependent cellular toxicity (ADCC) of antibodies produced in sugar chain-modified cell lines.

### DETAILED DESCRIPTION OF THE INVENTION

The present invention provides a method for reducing the fucose content of a recombinant protein, which comprises expressing in an animal cell the recombinant protein and one or more proteins selected from the group consisting of:

a) FUCA1 having the amino acid sequence of SEQ ID NO: 6;

b) an FUCA1 mutant having an amino acid sequence obtained by replacing asparagine of the amino acid sequence of FUCA1 with other amino acid;

c) FUCA2 having the amino acid sequence of SEQ ID NO: 7; and

d) a fragment of the localization domain of FUT8 having the amino acid sequence of SEQ ID NO: 1.

In the present invention, there is provided a method for reducing the fucose content of a therapeutic antibody obtained from a conventional antibody-producing animal cell line, which comprises the step of modifying the antibody-producing animal cell line by glyco-engineering using fucosidase hydrolyzing alpha-1,6-fucose, which binds to the asparagine residue of the N-acethyl glucosamine sugar chain structure of antibody Fc region, and/or alpha-1,6-fucosyltransferase involved in the synthesis of alpha-1,6-fucose.

In the present invention, the animal cell is first transfected with an expression vector for the recombinant protein and then one or more proteins selected from the group consisting of a) to d) are expressed therein.

In the method of the present invention, the “expression vector” for the recombinant protein can be introduced into an animal cell after overexpressing one or more proteins selected from the group consisting of a) to d) in the animal cell.

In the present invention, the procedure of expressing one or more of the proteins a) to d) is conducted by introducing into the animal cell i) a recombinant vector comprising a DNA encoding the proteins or ii) recombinant vectors each comprising a DNA encoding any one of the proteins a) to d).

In the present invention, the “recombinant protein” is preferably a glycoprotein having alpha-1,6-fucose on the N-acetylglucosamine reducing sugar terminal of the glycoprotein's carbohydrate moiety. Further, representative examples of the recombinant protein include antibodies, and the antibodies may be human IgGs such as IgG1, IgG2, IgG3 and IgG4; or chimeric, humanized or human antibodies.

In the present invention, the “animal cell” may be one of the known animal cells capable of producing a glycoprotein including an antibody, and representative examples of the “animal cell” include but not limited to the cells of CHO (Chinese hamster ovary), rat myeloma, BHK (Baby hamster kidney), hybridoma, Namalwa, embryonic stem and fertilized egg.

In the present invention, “FUCA1 ” and “FUCA2” may be proteins having the amino acid sequences of SEQ ID NOs: 6 and 7, respectively.

In the present invention, the “FUCA1 mutant” may be a protein having an amino acid sequence obtained by replacing the 263^{rd} asparagine of FUCA1 having the amino acid sequence of SEQ ID NO: 6 with other amino acid, e.g., valine.

In the present invention, the “fragment of FUT8 localization domain” (or “fragment of the localization domain of FUT8” may be a protein obtained by deleting the catalytic domain from wild type FUT8, which can reduce the content of fucose attached to antibody Fc regions by inhibiting the FUT8 retention in the Golgi apparatus or repressing FUT8 activity by inducing an inappropriate FUT8 aggregate, when overexpressed in a cell line expressing antibody. The fragment of FUT8 localization domain may comprise the cytoplasmic tail (CT), the transmembrane domain (TM) or the stem region (stem), which is known to be involved in the localization and retention of FUT8 in Golgi apparatus. Consequently, the fragment of FUT8 localization domain may be a protein having an amino acid sequence consisting of the 1 to n^{th} amino acids of FUT8 having the amino acid sequence of SEQ ID NO: 1, wherein n is an integer ranging from 30 to 200. Preferred examples of the fragment of FUT8 localization domain include a fragment of SEQ ID NO: 2 wherein n is 30 (comprising CT and TM in FUT8), a fragment of SEQ ID NO: 3 wherein n is 101 (comprising CT, TM and a part of stem in FUT8), a fragment of SEQ ID NO: 4 wherein n is 125 (comprising CT, TM and a part of stem in FUT8; “stem 1”) and a fragment of SEQ ID NO: 5 wherein n is 200 (comprising CT, TM and a part of stem in FUT8; “stem 2”).

Further, the present invention provides a method for reducing the fucose content of a recombinant protein, which comprises expressing in an animal cell the recombinant protein and a fusion protein obtained by fusing a fragment of the localization domain of FUT8 having the amino acid sequence of SEQ ID NO: 1 with a protein selected from the group consisting of:

a) a fragment of FUCA1, which has the amino acid sequence obtained by deleting 1^{st} to 26^{th} amino acids of the amino acid sequence of SEQ ID NO: 6;

b) a fragment of FUCA2, which has the amino acid sequence obtained by deleting 1^{st} to 28^{th} amino acids of the amino acid sequence of SEQ ID NO: 7; and

c) a mutant of FUCA1 fragment, which has the amino acid sequence obtained by replacing asparagine of the amino acid sequence of the fragment of FUCA1 with other amino acid.

In the method of the present invention, the animal cell is first transfected with an expression vector for the recombinant protein and then one or more proteins selected from the group consisting of a) to c) are expressed therein.

In the method of the present invention, the “expression vector” for the recombinant protein may be introduced into the animal cell after expressing the fusion protein of the fragment of FUT8 localization domain and a fragment selected from the group consisting of a) to c) in the animal cell.

In the present invention, the “fragment of FUCA1 ” (or “FUCA1 fragment”) and the “fragment of FUCA2” (or “FUCA2 fragment”) may be proteins obtained by deleting signal sequences from FUCA1 and FUCA2 having the amino acid sequences of SEQ ID NOs: 6 and 7, respectively.

In the present invention, the “mutant of FUCA1 fragment” may be a protein having the amino acid sequence obtained by replacing asparagine of the fragment of FUCA1 with other amino acid, e.g., valine. In one embodiment of the present invention, the mutant of FUCA1 fragment may be a protein having the amino acid sequence obtained by replacing 263^{rd} asparagine with other amino acid, e.g., valine, and deleting 1^{st} to 26^{th} amino acids from the FUCA1 amino acid sequence of SEQ ID NO: 6. Such modification can make fucosidase lose its lysosome-targeting property so as to be localized to other intracellular regions such as Golgi apparatus.

In the present invention, the fusion protein may be a protein obtained by fusing the catalytic domain of FUCA1, FUCA2 or FUCA1 mutant without the signal sequence thereof with the fragment of FUT8 localization domain described above. Representative examples of the fusion protein include a fusion protein prepared by fusing a fragment of FUT8 localization domain having the amino acid sequence of SEQ ID NO: 3 with a FUCA1 fragment having the amino acid sequence obtained by deleting 1^{st} to 26^{th} amino acids from the amino acid sequence of SEQ ID NO: 6; a fusion protein prepared by fusing a fragment of FUT8 localization domain having the amino acid sequence of SEQ ID NO: 3 with a FUCA2 fragment having the amino acid sequence obtained by deleting 1^{st} to 28^{th} amino acids from the amino acid sequence of SEQ ID NO: 7; a fusion protein prepared by fusing a fragment of FUT8 localization domain having the amino acid sequence of SEQ ID NO: 2 with a mutant of FUCA1 fragment, which has the amino acid sequence obtained by replacing the 263^{rd} aspargine with valine and deleting 1^{st} to 26^{th} amino acids from the amino acid sequence of SEQ ID NO: 6; and a fusion protein prepared by fusing a fragment of FUT8 localization domain having the amino acid sequence of SEQ ID NO: 3 with a mutant of FUCA1 fragment, which has the amino acid sequence obtained by replacing the 263^{rd} aspargine with valine and deleting 1^{st} to 26^{th} amino acids from the amino acid sequence of SEQ ID NO: 6.

The recombinant protein, the animal cell and the fragment of FUT8 localization domain are the same as described above, respectively.

In the method of the present invention, the procedure of expressing the fusion protein is conducted by introducing into the animal cell i) a recombinant vector comprising a DNA encoding the fusion proteins or ii) recombinant vector each comprising a DNA encoding anyone of the fusion proteins.

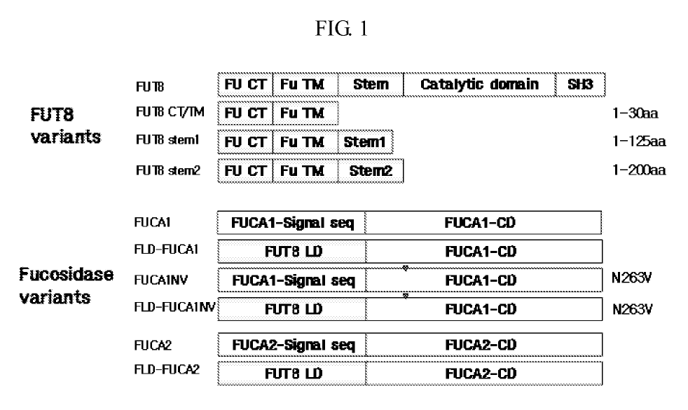

Gene structures of FUT8 and the fragments of FUT8 localization domain; FUCA1, FUCA2 and FUCA1 mutant; and the inventive fusion proteins, used for the modification of sugar chain in the present invention, are shown in FIG. 1.

In the present invention, the “recombinant protein” is preferably a glycoprotein having alpha-1,6-fucose in its structure, such as an antibody. Alpha-1,6-fucose has a feature of existing on the N-acetylglucosamine reducing sugar terminal of the glycoprotein's carbohydrate moiety. When the recombinant protein is an antibody, the antibody expressed according to the method of the present invention exhibits a reduced fucose content in its Fc region, which leads to the improvement in the therapeutic activity thereof.

The following Examples are intended to further illustrate the present invention without limiting its scope.

### EXAMPLE 1

### Construction of Expression Vectors for FUCA1, FUCA2 and FUCA1 Mutant

In order to construct expression vectors for FUCA1, FUCA2 and FUCA1 mutant, cDNAs encoding FUCA1, FUCA2 and FUCA1 mutant were each prepared by RT-PCR using human liver total RNA (Clontech, cat.636531, lot. 5070344) as a template, and cloned into pcDNA3.1 B(−)Myc-His vector (Invitrogen, INw-V855-20, US; hereinafter, referred to as “pcDNA”) using restriction sites of NheI (NEB,131L)/XhoI(NEB, 146L).

Specifically, a cDNA was synthesized from the human liver total RNA using Superscript first-strand synthesis kit (Invitrogen, 11904-018), and whole sequences encoding FUCA1 and FUCA2 were obtained by employing the synthesized cDNA as a template and primer pairs of SEQ ID NOs: 8 and 9 and SEQ ID NOs: 10 and 11, respectively. pcDNA (Invitrogen) and the whole sequences encoding FUCA1 or FUCA2 were digested with NheI and XhoI restriction enzymes, and the resulting DNAs were ligated to each other using DNA ligation kit (TAKARA, 6023). E. coli (DH5α) cells were transformed with the resulting ligated DNAs and the vectors containing FUCA1 and FUCA2 DNAs were isolated and designated pcDNA-FUCA1 and pcDNA-FUCA2, respectively. The sequences of the vectors thus obtained were confirmed through sequence analysis.

FUCA1 mutant (“FUCA1NV”) is a protein obtained by replacing the 263^{rd} amino acid, i.e., asparagine, of FUCA1 with valine. A cDNA encoding FUCA 1 mutant was obtained by amplifying its 5′ fragment and 3′ fragment using primer pairs of SEQ ID NOs: 8 and 12 and SEQ ID NOs: 9 and 13, respectively, and performing overlap PCR using primers of SEQ ID NOs: 8 and 9. In the same manner as described above, the cDNA encoding FUCA1 mutant was inserted into pcDNA (Invitrogen) using NheI/XhoI restriction sites to obtain pcDNA-FUCA1NV.

Plasmids designated pcDNA-FUCA1, pcDNA-FUCA2 and pcDNA-FUCA1NV were each harvested by using Endofree plasmid maxi kit (Quigen, 12362).

### EXAMPLE 2

### Construction of Expression Vectors for Fragments of FUT8 Localization Domain

A cDNA encoding FUT8 was prepared by RT-PCR using human liver total RNA as a template, and cloned in pcDNA vector (Invitrogen) using NheI/XhoI restriction sites.

Specifically, a cDNA was synthesized from the human liver total RNA using Superscript first-strand synthesis kit, and whole sequence encoding FUT8 was obtained by employing the synthesized cDNA as a template and primer pairs of SEQ ID NOs: 14 and 15. pcDNA (Invitrogen) and the whole sequence encoding FUT8 were digested with NheI and XhoI restriction enzymes, and the resulting DNAs were ligated to each other using DNA ligation kit. E. coli (DH5α) cells were transformed with the resulting ligated DNAs and the vector containing FUT8 DNA were isolated and designated pcDNA-FUT8. The sequences of the vectors thus obtained were confirmed through sequence analysis, and the plasmid DNA was harvested by using Endofree plasmid maxi kit.

DNAs encoding fragments of FUT8 localization domain, which have amino acid sequences of 1^{st} to 30^{th} (CT/TM; SEQ ID NO: 2), 1^{st} to 125^{th} (stem1; SEQ ID NO: 4) and 1^{st} to 200^{th} (stem2; SEQ ID NO: 5) amino acids of FUT8, respectively, were amplified using pcDNA-FUT8 as a template and primer pairs of SEQ ID NOs: 14 and 16, SEQ ID NOs: 14 and 17 and SEQ ID NOs: 14 and 18, respectively. Then, in the same manner as described above, the amplified DNAs were each cloned in pcDNA to obtain pcDNA-FUT8-CT/TM, pcDNA-FUT8-stem1 and pcDNA-FUT8-stem2.

### EXAMPLE 3

### Construction of Expression Vectors for Fusion Proteins Between a Fragment of FUT8 Localization Domain and the Catalytic Domain of FUCA1, FUCA2 or FUCA1 Mutant

DNAs encoding fusion proteins between a fragment of FUT8 localization domain and the catalytic domain of FUCA1, FUCA2 or FUCA1 mutant (without the signal sequence of FUCA1, FUCA2 or FUCA1 mutant) were prepared by performing overlap PCR using a DNA encoding a fragment of FUT8 localization domain and a DNA encoding a fragment of FUCA1, or FUCA2 or FUCA1 mutant, respectively.

Specifically, 5′ fragment of a DNA encoding a fusion protein between a fragment of FUT8 localization domain having the amino acid sequence of SEQ ID NO: 3 (i.e., 1^{st} to 101^{st} amino acids of FUT8) and a fragment of FUCA1 (i.e., a fragment consisting of 27^{th} to 461^{st} amino acids of FUCA1) (hereinafter, the fusion protein is referred to as “FLD-FUCA1”) was amplified by PCR using pcDNA-FUT8 as a template and primers of SEQ ID NOs: 14 and 19, and 3′ fragment of the DNA encoding FLD-FUCA1 was amplified by employing pcDNA-FUCA1 as a template and primers of SEQ ID NOs: 9 and 20. Overlap PCR was performed using the resulting DNA fragments and primers of SEQ ID NOs: 14 and 9 to obtain a DNA encoding FLD-FUCA1. The thus obtained DNA was cloned in pcDNA using NheI/XhoI restriction sites to obtain pcDNA-FLD-FUCA1.

Meanwhile, 5′ fragment of a DNA encoding a fusion protein between a fragment of FUT8 localization domain having the amino acid sequence of SEQ ID NO: 3 (i.e., 1^{st} to 101^{st} amino acids of FUT8) and a fragment of FUCA2 (i.e., a fragment consisting of 29^{th} to 467^{th} amino acids of FUCA2) (hereinafter, the fusion protein is referred to as “FLD-FUCA2”) was amplified by PCR using pcDNA-FUT8 as a template and primers of SEQ ID NOs: 14 and 21, and 3′ fragment of the DNA encoding FLD-FUCA2 was amplified by employing pcDNA-FUCA2 as a template and primers of SEQ ID NOs: 11 and 22. Overlap PCR was performed using the resulting DNA fragments and primers of SEQ ID NOs: 14 and 11 to obtain a DNA encoding FLD-FUCA2. The thus obtained DNA was cloned in pcDNA using NheI/XhoI restriction sites to obtain pcDNA-FLD-FUCA2.

Further, 5′ fragment of a DNA encoding a fusion protein between a fragment of FUT8 localization domain having the amino acid sequence of SEQ ID NO: 2 (i.e., 1^{st} to 30^{th} amino acids of FUT8) and a fragment of FUCA1 mutant (i.e., a fragment consisting of 27^{th} to 461^{st} amino acids of FUCA1 mutant) (hereinafter, the fusion protein is referred to as “FLD1-FUCA1NV”) was amplified by PCR using pcDNA-FUT8 as a template and primers of SEQ ID NOs: 14 and 23, and 3′ fragment of the DNA encoding FLD1-FUCA1NV was amplified by employing pcDNA-FUCA1NV as a template and primers of SEQ ID NOs: 9 and 24; and 5′ fragment of a DNA encoding a fusion protein between a fragment of FUT8 localization domain having the amino acid sequence of SEQ ID NO: 3 (i.e., 1^{st} to 101^{st} amino acids of FUT8) and a fragment of FUCA1 mutant (i.e., a fragment consisting of 27^{th} to 461^{st} amino acids of FUCA1 mutant) (hereinafter, the fusion protein is referred to as “FLD2-FUCA1NV”) was amplified by PCR using pcDNA-FUT8 as a template and primers of SEQ ID NOs: 14 and 19, and 3′ fragment of the DNA encoding FLD2-FUCA1NV was amplified by employing pcDNA-FUCA1NV as a template and primers of SEQ ID NOs: 9 and 20. Then, overlap PCR was performed using the resulting DNA fragments and primers of SEQ ID NOs: 14 and 9 to obtain DNAs encoding FLD1-FUCA1NV and FLD2-FUCA1NV, which were cloned in pcDNA using NheI/XhoI restriction sites to obtain pcDNA-FLD1-FUCA1NV and pcDNA-FLD2-FUCA1NV, respectively.

The thus obtained plasmids designated pcDNA-FLD-FUCA1, pcDNA-FLD-FUCA2, pcDNA-FLD1-FUCA1NV and pcDNA-FLD2-FUCA1NV were each harvested by using Endofree plasmid maxi kit (Quigen, 12362).

### EXAMPLE 4

### Preparation of Control Antibody-Expressina Cell Line using siRNA

A cell line expressing control antibody exhibiting a reduced fucose content was prepared by introducing FUT8 siRNA into a cell line expressing a therapeutic antibody.

Specifically, a DNA encoding a control antibody, i.e., Rituxan, which is an anti-CD20 therapeutic antibody (U.S. Pat. No. 5,736,137) was inserted into pMSG vector (Korean Patent No. 408844; KCCM 10202), and CHO-DG44 (dhfr-) cells (Dr. Lawrence Chasin, Columbia University, New York, USA) were transfected with the resulting plasmid and pDCH1P plasmid (Dr. Lawrence Chasin, Columbia University, New York, USA) expressing dihydrofolate reductase (DHFR). After harvesting the transfected colonies, the cells were adapted to gradually increasing concentrations (until 1 μM) of methotrexate (MTX), which is a folate analog, to obtain CHO-Rituxan cell line highly expressing Rituxan.

A cell line expressing control antibody exhibiting a reduced fucose content in its Fc region was prepared as follows, by employing two conventional siRNA sequences (Mori, K. et al., Biotechnology and Bioengineering, 88(7), 901-908, 2004).

In order to prepare B form (FUT8 siB) known to inhibit the region of SEQ ID NO: 25 in the whole nucleotide sequence of FUT8, primers of SEQ ID NOs: 26 and 27 were ligated to each other; and in order to prepare R form (FUT8 siR) known to inhibit the region of SEQ ID NO: 28 in the whole nucleotide sequence of FUT8, primers of SEQ ID NOs: 29 and 30 were ligated to each other. The ligated primers were each cloned in pSilencer 2.1-U6 hygro vector (Ambion, 5760) and transfected into CHO-Rituxan cells in the same manner as described above. The transfected cells were subjected to a primary selection using hygromycin as a selection marker, followed by final selection through FACS (fluorescence activated cell sorting) analysis using LCA (biotilated-lens culinaris agglutinin; Vector Lab, B-1045(S0925)) and phycoerythrin (PE) streptavidin (Vector Lab, SA-5007(R1209)) to obtain CHO-R-siRNA cell lines.

### EXAMPLE 5

### Preparation of Cell Lines Expressing Therapeutic Antibody with Modified Sugar Chain

For an example, CHO-Rituxan cells expressing the antibody of Rituxan were transfected with the plasmids comprising sugar chain modifying genes, pcDNA-FUT8, pcDNA-FUT8-CT/TM, pcDNA-FUT8-stem1, pcDNA-FUT8-stem2, pcDNA-FUCA1, pcDNA-FUCA2, pcDNA-FUCA1NV, pcDNA-FLD-FUCA1, pcDNA-FLD-FUCA2, pcDNA-FLD1-FUCA1NV and pcDNA-FLD2-FUCA1NV, obtained in Examples 1 to 3 by using lipofectamine 2000 (Invitrogen, 11668-019(1369361)), and the recombinant cell lines were each selected using neomycin as a selection marker. The cell lines thus obtained were designated CHO-R-FUT8, CHO-R-FUT8-CT/TM, CHO-R-FUT8-stem1, CHO-R-FUT8-stem2, CHO-R-FUCA1, CHO-R-FUCA2, CHO-R-FUCA1NV, CHO-R-FLD-FUCA1, CHO-R-FLD-FUCA2, CHO-R-FLD1-FUCA1NV and CHO-R-FLD2-FUCA1NV, respectively.

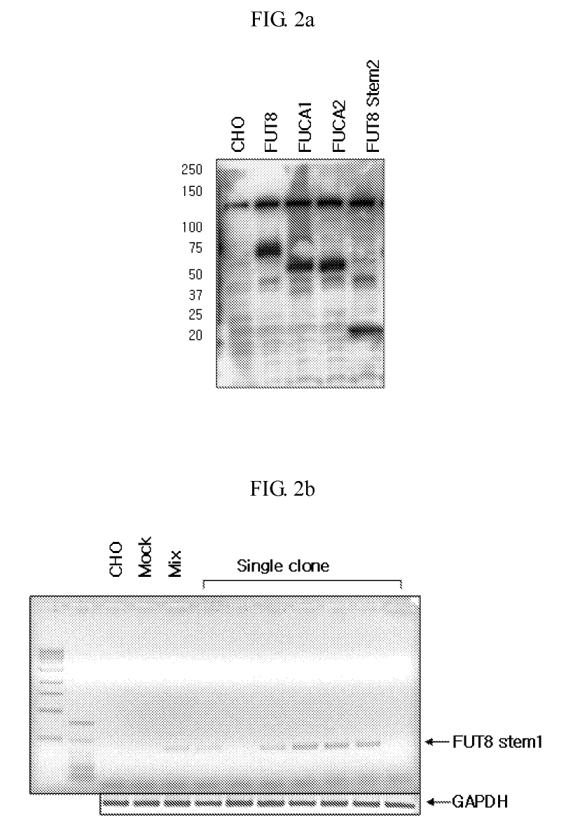

In order to select CHO cell lines highly expressing sugar chain modifying genes, the cell lines thus obtained were each subjected to western blot analysis using rabbit anti-His antibody (SantaCruz, His-probe (G-18) rabbit polyclonal, sc804 (H3006)) and Goat anti-rabbit antibody (KPL, Goat anti-rabbit IgG(H+L)−HRP, 074-1506), and to RT-PCR analysis, and the results are shown in FIGS. 2A and 2B.

As shown in FIG. 2A, the sugar chain modifying gene of FUT8, FUCA1 or FUCA2 mutant was over-expressed in the CHO cell lines. Further, as shown in FIG. 2B, the sugar chain modifying gene of FUT8-stem1 was over-expressed in the individually isolated subdlones of the FUT8-stem1 CHO cell lines. The lane mix is transfectants comprising mixed isolated subdlones of FUT8-stem1; the lane CHO is CHO DG44 cell line; and mock is a transfectant prepared using only vector.

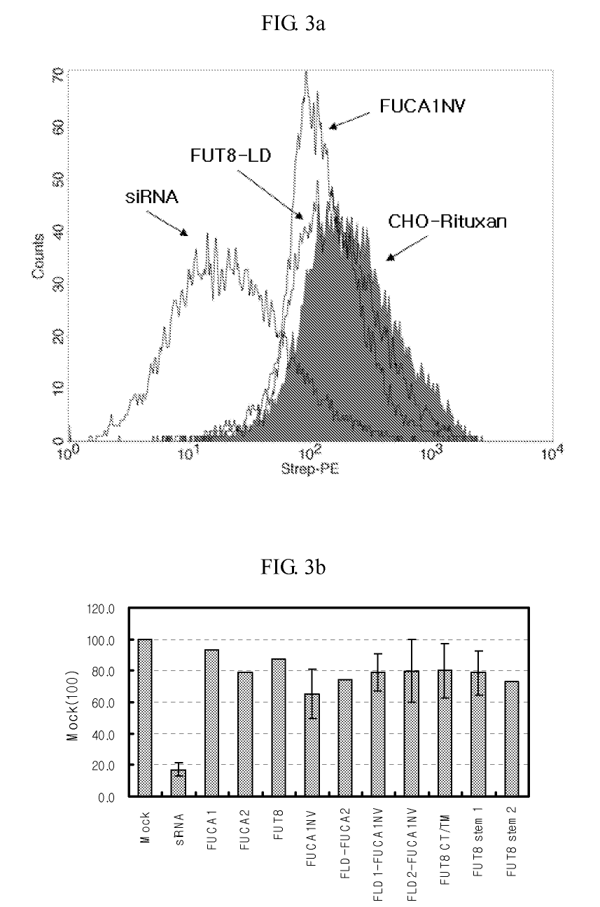

In addition, the results from FACS analysis using LCA (Vector Lab, B-1045(S0925)) and phycoerythrin streptavidin (Vector Lab, SA-5007(R1209)) are shown in FIGS. 3A and 3B.

As shown in FIG. 3A, the inventive cell lines exhibited low affinities to LCA, as compared with CHO-Rituxan cells lines. Further, as shown in FIG. 3B, the relative FACS mean values of the inventive cell lines were reduced by about 10˜35% from that of CHO-Rituxan cells, the values being, CHO-R-siRNA: 0.17, CHO-R-FUCA1: 0.93, CHO-R-FUCA2: 0.79, CHO-R-FUT8: 0.87, CHO-R-FUCA1NV: 0.65, CHO-R-FLD-FUCA2: 0.74, CHO-R-FLDIFUCA1NV: 0.79, CHO-R-FLD2-FUCA1NV: 0.80, CHO-R-FUT8-CT/TM: 0.80, CHO-R-FUT8-stem1: 0.79 and CHO-R-stem2: 0.73.

These results suggest that FUT8 localization domain, FUCA1, FUCA2, FUCA1 mutant, and a fusion protein of a fragment of FUT8 localization domain and a fragment of FUCA1, FUCA2 or FUCA1 mutant can be advantageously used for reducing fucose contents in sugar chain structures of glycoproteins.

### EXAMPLE 6

### Analysis for Sugar Chains of Antibodies

Antibodies produced by the cell lines prepared in Example 5, which expresses therapeutic antibody with modified sugar chain, were purified and subjected to a monosaccharide analysis using BIO-LC system (DC ICS 3000 system, DIONEX, 06110276) to quantitatively analyze the sugar chains in the Fc regions thereof as follows.

In embodiment, each cultured medium of the cell lines was purified using a Protein G Sepharose (Amersham, 17-0618-01) column. In this purification, 20 mM sodium phosphate buffer (pH 7.0) was employed as a binding buffer; 0.5 M glycine (pH 2.7), as a diluting buffer; and 1 M Tris-HCl (pH 9.0), as a neutralizing buffer. The purity of each purified antibody was analyzed by silver staining.

Each purified antibody was heated at 100° C. for 4 hrs in 4 M TFA (Trifluoroacetic acid) to separate monosaccharides therefrom, and dried in a vacuum dryer. The resulting residues were dissolved in deionized water and analyzed using BIO-LC system to determine fucose contents. The BIO-LC system was equipped with ED detector (DIONEX, 06110046), amino trap column (DIONEX, 046122) as a guard column and CarboPac PA 10 column (DIONEX, 046110) as an analysis column.

Each sample of 25 μl was used in analyzing the monosaccharides, and DI water and 200 mM NaOH (Fisher, SS254-1) were employed as eluents. The analysis conditions and the time-dependent changes in the concentration of the eluents are shown in Tables 1 and 2, respectively.

TABLE 1
Analysis conditions

| | |
|---|---|
| Amount of sample | 25 μl |
| Analysis column | CarboPac PA 10 (4 × 250 mm) |
| Guard column | Amino-trap column (4 × 50 mm) or PA 10 |
| Detector | ED detector |
| Eluent A | Deionized water |
| Eluent B | 200 mM NaOH |

TABLE 2

| Time | Concentration of | Concentration of | |
|---|---|---|---|
| (min) | eluent A (%) | eluent B (%) | Condition |
| 0 | 91 | 9 | 18 mM NaOH |
| 20 | 91 | 9 | 18 mM NaOH |
| 20.1 | 0 | 100 | Washing condition |
| 25 | 0 | 100 | Washing condition |
| 25.1 | 91 | 9 | Re-equilibration condition |
| 30 | 91 | 9 | Re-equilibration condition |

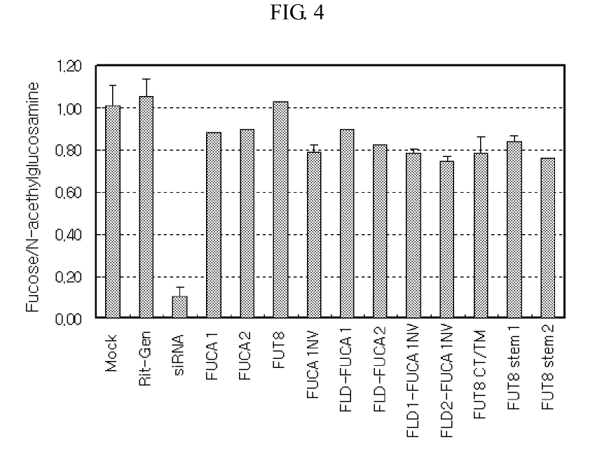

The content of monosaccarides was represented as ratios of fucose to four N-acetylglucosamines existed in the frame of sugar chain of antibody Fc region. As shown in FIG. 4, the inventive cell lines exhibited reduced ratio of fucose/N-acetylglucosamine by about 10-25% as compared with that of CHO-Rituxan cell lines, the ratios being, CHO-Rituxan: 1.01, CHO-R-siRNA: 0.10, CHO-R-FUCA1: 0.88, CHO-R-FUCA2: 0.90, CHO-R-FUT8: 1.03, CHO-R-FUCA1NV: 0.75, CHO-R-FLD-FUCA1: 0.90, CHO-R-FLD-FUCA2: 0.82, CHO-R-FLD1-FUCA1NV: 0.79, CHO-R-FLD2-FUCA1NV: 0.75, CHO-R-CT/TM: 0.78, CHO-R-FUT8-stem1: 0.84 and CHO-R-FUT8-stem2: 0.76.

Similarly, the fragments of FUT8 localization domain reduced the fucose contents in antibody Fc regions by about 15 to 25% when introduced into therapeutic antibody-expressing cell lines. Further, the FUCA1 or FUCA2-overexpressing cell lines of the present invention exhibited reduced fucose contents by about 10%, and the inventive cell lines overexpressing a FUCA1 mutant, i.e., FUCA1NV, exhibited reduced fucose contents by about 25%. Furthermore, the inventive cell lines expressing fusion proteins of a fragment of FUT8 localization domain and a catalytic domain of FUCA1, FUCA2 or FUCA1 mutant exhibited reduced fucose contents by 10˜25%.

These results suggest that the inventive antibody-expressing cell lines overexpressing fragments of FUT8 localization domain, FUCA1, FUCA2, FUCA1 mutant, and fusion proteins of a fragment of FUT8 and a fragment of FUCA1, FUCA2 or FUCA1 mutant can be advantageously used for producing therapeutic antibodies having low fucose contents.

### EXAMPLE 7

### Analysis for the Therapeutic Effects of the Antibodies with a Modified Sugar Chain

The therapeutic effects of the inventive antibodies having modified sugar chain were analyzed by determining the complement-dependent cytotoxicities (CDC), the binding affinity with Fc region receptor and the antibody-dependent cellular cytotoxicities (ADCC) thereof.

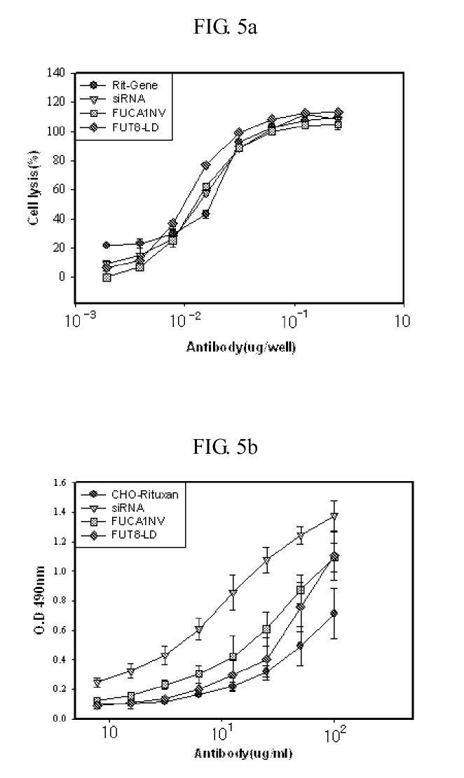

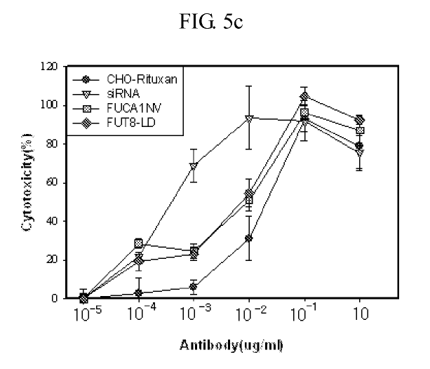

Specifically, for the CDC test, CD20-expressing Daudi (ATCC CCL-213) B-cell lymphoma cells were treated with anti-CD20 antibodies purified from CHO-Rituxan, CHO-R-siRNA, CHO-R-FUT8-LD (CHO-R-FUT8-CT/TM and CHO-R-FUT8-stem1) and CHO-R-FUCA1NV cells, and standard human plasma (DADE Behring), respectively, and then, treated with WST-1 reagent (Roche), followed by determining the absorbance intensities of the cells at 450 nm and 690 nm (Hodoniczky J. et al., Biotechnology Progress, 21(6), 1644-1652, 2005). As shown in FIG. 5A, the antibodies purified from the inventive cell lines exhibited similar CDC values.

For measuring binding capacity of the antibody Fc region with an Fc receptor, an antibody Fc region receptor (FcrRIIIa) was obtained from human peripheral blood mononuclear cell (PBMC) using RT-PCR (Clémenceau, B. et. al., Blood, 107(12), 4669-4677, 2006). Specifically, in order to clone a gene encoding soluble antibody Fc region receptor (FcrRIIIa) (GenBank, X52645), total RNA was isolated from human PBMC, and subjected to RT-PCR using cDNA synthesis kit (Invitrogen, 18080-051) to obtain cDNA. PCR was performed using the obtained cDNA as a template and primers of SEQ ID NOs: 31 and 32 to obtain a DNA encoding FcrRIIIa-His, wherein the cytoplasmic tail domain and transmembrane domain were deleted from FcrRIIIa and 6 histidine (His) residues were attached to the extracellular domain following signal sequence at the 5′ end thereof. FcrRIIIa-His DNA was cloned in pMSG vector to obtain a plasmid designated pMSG-FcrRIIIa-his. Plasmid pMSG-FcrRIIIa-his was transfected into CHO cells using lipofectamine 2000 (Invitrogen, 11668-019), and the expression of FcrRIIIa was amplified through MTX system. The finally selected CHO-FcrRIIIa-his cell line was incubated in a conventional cell culture medium, and 200 ml of the incubated medium was subjected to His column (Novagen) purification to obtain purified FcrRIIIa-His. The binding capacity of antibody Fc region with the Fc receptor was measured by ELISA using anti-His monoclonal antibody (QIAGEN, anti-4X his antibody) and anti-human F(ab)2-HRP (Jackson Lab, 309-036-006).

As shown in FIG. 5B, the absorbance values of the test cell lines were increased in an antibody dose-dependent manner, the inventive cell lines, and Rituxan having modified sugar chain exhibited higher binding affinity to FcrRIIIa-His by about 4 folds as compared with normal Rituxan.

For the ADCC (Antibody-dependent cellular toxicity) test, Daudi (ATCC CCL-213) cells (1×10⁴ cells/well) expressing CD20 were treated with Cr-51 to introduce Cr-51 thereinto, and then treated with purified antibodies produced from CHO-Rituxan, CHO-R-siRNA, CHO-R-FUT8-CT/TM, CHO-R-FUT8-stem1 and CHO-R-FUCA1NV cell lines and PBMC (4×10⁵ cells) isolated from the bloods of healthy volunteers, followed by measuring the amount of Cr-51 released from dead cells using a gamma counter. The ADCC of each antibody was calculated from the measured values using the following mathematical formula (Shinkawa, T. et al., Journal of Biological Chemistry, 278(5), 3466-3473, 2003):

ADCC(%)=100×(E−ST)/(M−ST)
E: experimental release
ST: spontaneous release
M: maximum release

As shown in FIG. 5C, the antibodies having modified sugar chains produced from the inventive cell lines exhibited ADCC values increased by about 2 to 5 folds as compared with that of antibody having normal sugar chains.

While the invention has been described with respect to the above specific embodiments, it should be recognized that various modifications and changes may be made to the invention by those skilled in the art which also fall within the scope of the invention as defined by the appended claims.

What is claimed is:

1. A method for reducing the fucose content of a recombinant protein, which comprises expressing in an animal cell the recombinant protein and one or more proteins selected from the group consisting of:
a) FUCA1 having the amino acid sequence of SEQ ID NO: 6;
b) an FUCA1 mutant having an amino acid sequence obtained by replacing asparagine of the amino acid sequence of FUCA1 with other amino acid;
c) FUCA2 having the amino acid sequence of SEQ ID NO: 7; and
d) a fragment of the localization domain of FUT8 having the amino acid sequence of SEQ ID NO: 1.

2. The method of claim 1, wherein the recombinant protein is a glycoprotein having alpha-1,6-fucose.

3. The method of claim 2, wherein the alpha-1,6-fucose exists on the N-acetylglucosamine reducing sugar terminal of the glycoprotein's carbohydrate moiety.

4. The method of claim 1, wherein the recombinant protein is an antibody.

5. The method of claim 4, wherein the fucose content of the antibody is reduced to elevate the therapeutic effect of the antibody.

6. The method of claim 1, wherein the animal cell is first transfected with an expression vector for the recombinant protein and then one or more proteins selected from the group consisting of a) to d) are expressed therein.

7. The method of claim 1, wherein an expression vector for the recombinant protein is introduced into the animal cell after expressing one or more proteins selected from the group consisting of a) to d) in the animal cell.

8. The method of claim 1, wherein the fragment of FUT8 localization domain has an amino acid sequence composed of the 1^{st} to n^{th} amino acids of FUT8 having the amino acid sequence of SEQ ID NO: 1, n being an integer ranging from 30 to 200.

9. The method of claim 8, wherein n is 30 and the fragment of FUT8 localization domain has the amino acid sequence of SEQ ID NO: 2.

10. The method of claim 8, wherein n is 125 and the fragment of FUT8 localization domain has the amino acid sequence of SEQ ID NO: 4.

11. The method of claim 8, wherein n is 200 and the fragment of FUT8 localization domain has the amino acid sequence of SEQ ID NO: 5.

12. The method of claim 1, wherein the FUCA1 mutant has the amino acid sequence obtained by replacing the 263^{rd} asparagine of FUCA1 having the amino acid sequence of SEQ ID NO: 6 with valine.

13. The method of claim 1, wherein the animal cell is selected from the group consisting of cells of CHO (Chinese hamster ovary), rat myeloma, BHK (baby hamster kidney), hybridoma, Namalwa, embryonic stem and fertilized egg.

14. The method of claim 1, wherein the procedure of expressing one or more of the proteins a) to d) is conducted by introducing into the animal cell i) a recombinant vector comprising a DNA encoding the proteins or recombinant vectors each comprising a DNA encoding any one of the proteins a) to d).

15. A method for reducing the fucose content of a recombinant protein, which comprises expressing in an animal cell the recombinant protein and a fusion protein obtained by fusing a fragment of the localization domain of FUT8 having the amino acid sequence of SEQ ID NO: 1 with a protein selected from the group consisting of:
a) a fragment of FUCA1, which has the amino acid sequence obtained by deleting 1^{st} to 26^{th} amino acids of the amino acid sequence of SEQ ID NO: 6;
b) a fragment of FUCA2, which has the amino acid sequence obtained by deleting 1^{st} to 28^{th} amino acids of the amino acid sequence of SEQ ID NO: 7; and
c) a mutant of FUCA1 fragment, which has the amino acid sequence obtained by replacing asparagine of the amino acid sequence of the fragment of FUCA1 with other amino acid.

16. The method of claim 15, wherein the recombinant protein is a glycoprotein having alpha-1,6-fucose.

17. The method of claim 16, wherein the alpha-1,6-fucose exists on the N-acetylglucosamine reducing sugar terminal of the glycoprotein's carbohydrate moiety.

18. The method of claim 15, wherein the recombinant protein is an antibody.

19. The method of claim 18, wherein the fucose content of the antibody is reduced to elevate the therapeutic effect of the antibody.

20. The method of claim 15, wherein the animal cell is first transfected with an expression vector for the recombinant protein and then one or more proteins selected from the group consisting of a) to c) are expressed therein.

21. The method of claim 15, wherein an expression vector for the recombinant protein is introduced into then animal cell after expressing the fusion protein of the fragment of FUT8 localization domain and a fragment selected from the group consisting of a) to c) in the animal cell.

22. The method of claim 15, wherein the fragment of FUT8 localization domain has an amino sequence composed of 1^{st} to nth amino acids of FUT8 having the amino acid sequence of SEQ ID NO: 1, n being an integer ranging from 30 to 200.

23. The method of claim 22, wherein n is 30 and the fragment of FUT8 localization domain has the amino acid sequence of SEQ ID NO: 2.

24. The method of claim 22, wherein n is 101 and the fragment of FUT8 localization domain has the amino acid sequence of SEQ ID NO: 3.

25. The method of claim 22, wherein n is 125 and the fragment of FUT8 localization domain has the amino acid sequence of SEQ ID NO: 4.

26. The method of claim 22, wherein n is 200 and the fragment of FUT8 localization domain has the amino acid sequence of SEQ ID NO: 5.

27. The method of claim 15, wherein the mutant of FUCA1 fragment has the amino acid sequence obtained by replacing the 263^{rd} asparagine with valine and deleting 1^{st} to 26^{th} amino acids from the amino acid sequence of SEQ ID NO: 6.

28. The method of claim 15, wherein the fusion protein is a protein prepared by fusing a fragment of FUT8 localization domain having the amino acid sequence of SEQ ID NO: 3 with a fragment of FUCA1 having the amino acid sequence obtained by deleting 1^{st} to 26^{th} amino acids from the amino acid sequence of SEQ ID NO: 6.

29. The method of claim 15, wherein the fusion protein is a protein prepared by fusing a fragment of FUT8 localization domain having the amino acid sequence of SEQ ID NO: 3 with a fragment of FUCA2 having the amino acid sequence obtained by deleting 1^{st} to 28^{th} amino acids from the amino acid sequence of SEQ ID NO: 7.

30. The method of claim 15, wherein the fusion protein is a protein prepared by fusing a fragment of FUT8 localization domain having the amino acid sequence of SEQ ID NO: 2 with a mutant of FUCA1 fragment having the amino acid sequence obtained by replacing the 263^{rd} asparagine with valine and deleting 1^{st} to 26^{th} amino acids from the amino acid sequence of SEQ ID NO: 6.

31. The method of claim 15, wherein the fusion protein is a protein prepared by fusing a fragment of FUT8 localization domain having the amino acid sequence of SEQ ID NO: 3 with a mutant of FUCA1 fragment having the amino acid sequence obtained by replacing the 263^{rd} asparagine with valine and deleting 1^{st} to 26^{th} amino acids from the amino acid sequence of SEQ ID NO: 6.

32. The method of claim 15, wherein the animal cell is selected from the group consisting of cells of CHO (Chinese hamster ovary), rat myeloma, BHK (baby hamster kidney), hybridoma, Namalwa, embryonic stem and fertilized egg.

33. The method of claim 15, wherein the procedure of expressing the fusion protein is conducted by introducing into the animal cell i) a recombinant vector comprising a DNA encoding the fusion proteins or ii) recombinant vector each comprising a DNA encoding anyone of the fusion proteins.


SEQUENCE LISTING (32 sequences)

SEQ ID NO: 1 - protein, 575 aa, Homo sapiens
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 2 - protein, 30 aa, Homo sapiens
MRPWTGSWRWIMLILFAWGTLLFYIGGHLV

SEQ ID NO: 3 - protein, 101 aa, Homo sapiens
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYK

SEQ ID NO: 4 - protein, 125 aa, Homo sapiens
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKE

SEQ ID NO: 5 - protein, 200 aa, Homo sapiens
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQN

SEQ ID NO: 6 - protein, 461 aa, Homo sapiens
MRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNSSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 7 - protein, 467 aa, Homo sapiens
MRPQELPRLAFPLLLLLLLLLPPPPCPAHSATRFDPTWESLDARQLPAWFDQAKFGIFIHWGVFSVPSFGSEWFWWYWQKEKIPKYVEFMKDNYPPSFKYEDFGPLFTAKFFNANQWADIFQASGAKYIVLTSKHHEGFTLWGSEYSWNWNAIDEGPKRDIVKELEVAIRNRTDLRFGLYYSLFEWFHPLFLEDESSSFHKRQFPVSKTLPELYELVNNYQPEVLWSDGDGGAPDQYWNSTGFLAWLYNESPVRGTVVTNDRWGAGSICKHGGFYTCSDRYNPGHLLPHKWENCMTIDKLSWGYRREAGISDYLTIEELVKQLVETVSCGGNLLMNIGPTLDGTISVVFEERLRQMGSWLKVNGEAIYETHTWRSQNDTVTPDVWYTSKPKEKLVYAIFLKWPTSGQLFLGHPKAILGATEVKLLGHGQPLNWISLEQNGIMVELPQLTIHQMPCKWGWALALTNVI

SEQ ID NO: 8 - DNA, 28 bp, Artificial Sequence
  forward primer for FUCA1, a Homo sapiens DNA sequence
ctagctagccaccatgaggtcgcggccg

SEQ ID NO: 9 - DNA, 35 bp, Artificial Sequence
  reverse primer for FUCA1, a Homo sapiens DNA sequence
ctactcgagcttcactcctgtcagctttatagtcc

SEQ ID NO: 10 - DNA, 28 bp, Artificial Sequence
  forward primer for FUCA2, a Homo sapiens DNA sequence
ctagctagccaccatgcggccccaggag

SEQ ID NO: 11 - DNA, 33 bp, Artificial Sequence
  reverse primer for FUCA2, a Homo sapiens DNA sequence
ctactcgaggatcacattagtcagggctagagc

SEQ ID NO: 12 - DNA, 36 bp, Artificial Sequence
  forward primer for FUCA1 variant, a  variant of Homo sapiens DNA sequence
ccatggtgacaggaacagacctgaccccatcggtca

SEQ ID NO: 13 - DNA, 36 bp, Artificial Sequence
  reverse primer for FUCA1 variant, a  variant of Homo sapiens DNA sequence
tgaccgatggggtcaggtctgttcctgtcaccatgg

SEQ ID NO: 14 - DNA, 29 bp, Artificial Sequence
  forward primer for FUT8, a Homo sapiens DNA sequence
ctagctagccaccatgcggccatggactg

SEQ ID NO: 15 - DNA, 31 bp, Artificial Sequence
  reverse primer for FUT8, a Homo sapiens DNA sequence
ctactcgagtttctcagcctcaggatatgtg

SEQ ID NO: 16 - DNA, 34 bp, Artificial Sequence
  primer for FUT8 localization domain fragment, a Homo sapiens DNA sequence; primer amplifies sequence corresponding to positions 1 to 30 of the protein sequence
ctactcgagtaccaagtgaccacctatataaaac

SEQ ID NO: 17 - DNA, 31 bp, Artificial Sequence
  primer for FUT8 localization domain fragment, a Homo sapiens DNA sequence; primer amplifies sequence corresponding to positions 1 to 125 of the protein sequence
ctactcgagctctttagctccattttcaatc

SEQ ID NO: 18 - DNA, 33 bp, Artificial Sequence
  primer for FUT8 localization domain fragment, a Homo sapiens DNA sequence; primer amplifies sequence corresponding to positions 1 to 200 of the protein sequence
ctactcgagattctgaagatatgttattctccg

SEQ ID NO: 19 - DNA, 43 bp, Artificial Sequence
  forward primer for FLD-FUCA1, a fusion of Homo sapiens DNA sequences
gtagcggcgcggaggctgcttgtaattttcaatctgttctttg

SEQ ID NO: 20 - DNA, 43 bp, Artificial Sequence
  reverse primer for FLD-FUCA1, a fusion of Homo sapiens DNA sequences
caaagaacagattgaaaattacaagcagcctccgcgccgctac

SEQ ID NO: 21 - DNA, 42 bp, Artificial Sequence
  forward primer for FLD-FUCA2, a fusion of Homo sapiens DNA sequences
aagcgcgtggcgctgtgcttgtaattttcaatctgttctttg

SEQ ID NO: 22 - DNA, 42 bp, Artificial Sequence
  reverse primer for FLD-FUCA2, a fusion of Homo sapiens DNA sequences
caaagaacagattgaaaattacaagcacagcgccacgcgctt

SEQ ID NO: 23 - DNA, 43 bp, Artificial Sequence
  forward primer for FLD-FUCA1NV, a fusion of Homo sapiens DNA sequences
gtagcggcgcggaggctgtaccaagtgaccacctatataaaac

SEQ ID NO: 24 - DNA, 43 bp, Artificial Sequence
  reverse primer for FLD-FUCA1NV, a fusion of Homo sapiens DNA sequences
gttttatataggtggtcacttggtacagcctccgcgccgctac

SEQ ID NO: 25 - DNA, 19 bp, Homo sapiens
gctgagtctctccgaatac

SEQ ID NO: 26 - DNA, 65 bp, Artificial Sequence
  forward primer for FUT8 siB, a Homo sapiens DNA sequence
gatccgctgagtctctccgaatacttcaagagagtattcggagagactcagccattttttggaaa

SEQ ID NO: 27 - DNA, 65 bp, Artificial Sequence
  reverse primer for FUT8 siB, a Homo sapiens DNA sequence
agcttttccaaaaaatggctgagtctctccgaatactctcttgaagtattcggagagactcagcg

SEQ ID NO: 28 - DNA, 22 bp, Homo sapiens
gaacactcatcatcttggaatc

SEQ ID NO: 29 - DNA, 65 bp, Artificial Sequence
  forward primer for FUT8 siR, a Homo sapiens DNA sequence
gatccgaacactcatcttggaatcttcaagagagattccaagatgagtgttcgcttttttggaaa

SEQ ID NO: 30 - DNA, 64 bp, Artificial Sequence
  reverse primer for FUT8 siR, a Homo sapiens DNA sequence
agcttttccaaaaaagcgaacactcatcttggaatctctcttgaagattccaagatgagtgttc

SEQ ID NO: 31 - DNA, 27 bp, Artificial Sequence
  forward primer for FcrRIIIa, a Homo sapiens DNA sequence
ctagctagctctttggtgacttgtcca

SEQ ID NO: 32 - DNA, 48 bp, Artificial Sequence
  reverse primer for FcrRIIIa, a Homo sapiens DNA sequence
ctagttaactcaatgatgatgatgatgatgcccaggtggaaagaatga